(19) United States
(12) Patent Application Publication
(10) Pub. No.: US 2013/0209760 A1
(43) Pub. Date: Aug. 15, 2013

(54) ALKYL SULFONATED TETRAZOLE COMPOUND, PREPARING METHOD THEREOF, AND EPOXY RESIN CONTAINING THE SAME, AND SUBSTRATE PRODUCED THEREFROM

(71) Applicants: Young Kwan SEO, Gyunggi-do (KR); Sung Nam CHO, Gyunggi-do (KR); Jun Young KIM, Gyunggi-do (KR); Tae Hoon KIM, Gyunggi-do (KR)

(72) Inventors: Young Kwan SEO, Gyunggi-do (KR); Sung Nam CHO, Gyunggi-do (KR); Jun Young KIM, Gyunggi-do (KR); Tae Hoon KIM, Gyunggi-do (KR)

(73) Assignee: SAMSUNG ELECTRO-MECHANICS CO., LTD., Gyunggi-do (KR)

(21) Appl. No.: 13/475,429

(22) Filed: May 18, 2012

(30) Foreign Application Priority Data
Feb. 15, 2012 (KR) 102012015428

(51) Int. Cl. C08G 59/14; B32B 15/08; B32B 3/10; C07D 257/04

(52) U.S. Cl. 428/209; 548/251; 525/523; 428/418

(57) ABSTRACT

The present invention provides an alkyl sulfonated tetrazole compound, a method of preparing the same, an epoxy resin containing the same and a substrate produced therefrom. The epoxy resin is usefully used as a raw material of printed circuit boards, and has high adhesion to metal.

### CROSS REFERENCE TO RELATED APPLICATION

This application claims the benefit of Korean Patent Application No. 10-2012-0015428, filed Feb. 15, 2012, entitled “Alkyl sulphonated tetrazole compound, preparing method thereof, and epoxy resin containing the same, and substrate produced therefrom”, which is hereby incorporated by reference in its entirety into this application.

### BACKGROUND OF THE INVENTION

1. Field of the Invention

The present invention relates to an alkyl sulfonated tetrazole compound, a method of preparing the same, an epoxy resin containing the same and a substrate produced therefrom.

2. Description of the Related Art

Generally, in electronic products such as printed circuit boards, methods of enhancing the adhesion or stickiness between a resin and a metal include a method of roughening the surface of a resin by desmearing and a method of mixing an additive for enhancing adhesion with a resin. The terms “stickiness”, “adhesion” and the like used in the present invention mean the bonding force between the resin and the metal.

Meanwhile, as the width of the wire decreases, the roughness of the surface of the resin must be low, so the effect of roughness on adhesion gradually becomes weak, with the result that an additive for enhancing adhesion, which is added to resin in order to enhance adhesion, plays an important part. EP 0,665,468 discloses a triazole-based or tetrazole-based additive which enhances the adhesion between a resin and a metal.

An additive for enhancing adhesion must be uniformly distributed in the resin in order to maximize the effect of enhancing the adhesion between the resin and the metal. Therefore, in order to uniformly disperse the additive in the resin, the additive may be dissolved in a solvent or may be introduced in the form of powder and then dispersed in the resin. However, these methods are problematic in terms of the compatibility between the solvent and the resin and the dispersion stability of the powder. Further, U.S. Pat. No. 4,322,459 discloses an epoxy resin, whose curing characteristics are improved by adding triazole or tetrazole. However, this patent document does not disclose or propose the improvement of adhesion.

### SUMMARY OF THE INVENTION

The present inventors found that, when an azole-derived thiol group (—S—) in an azole-substituted epoxy resin directly ring-opens an epoxy group of an epoxy compound and is connected thereto, the thiol group (—S—) cannot donate electrons to the epoxy group to such an extent that the electrons provide adhesion because of an hydroxy group (—OH) derived from the epoxy compound adjacent to the thiol group (—S—). Thus, they also found that an alkyl sulfonated tetrazole compound, which is prepared by alkyl-sulfonating tetrazole and bonding the alkyl-sulfonated tetrazole with epoxy, has very excellent adhesion to metal. Based on these findings, the present invention was completed.

Accordingly, the present invention has been made keeping in mind the above problems occurring in the related art, and the present invention is intended to provide an alkyl sulfonated tetrazole compound having high adhesion.

Further, the present invention is intended to provide a method of preparing the alkyl sulfonated tetrazole compound.

Further, the present invention is intended to provide an epoxy resin containing the alkyl sulfonated tetrazole compound, the epoxy resin having excellent adhesion to metal.

Further, the present invention is intended to provide a method of preparing the epoxy resin.

Further, the present invention is intended to provide an epoxy composition containing the epoxy resin.

Further, the present invention is intended to provide a prepreg produced from the epoxy composition.

Further, the present invention is intended to provide a film produced from the epoxy composition.

Further, the present invention is intended to provide a substrate produced from the epoxy composition.

In order to achieve the above objects, an aspect of the present invention provides an alkyl sulfonated tetrazole compound represented by Formula 1 below:

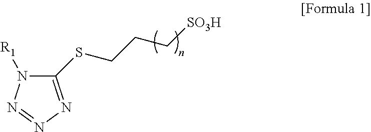

wherein R₁ is selected from an aliphatic or alicyclic alkyl group of 1 to 20 carbon atoms, an aryl or aralkyl group of 1 to 20 carbon atoms, an alkyl or aryl group substituted with a functional group of 1 to 20 carbon atoms, a ring linked with an alkylene group including or not including a heteroatom, a polymer compound group, and derivatives thereof; and n is an integer of 1 to 6.

Here, the alkyl sulfonated tetrazole compound may be 3-(1-methyl-1H-tetrazole-5-ylthio)propane-1-sulfonic acid or 3-(1-methyl-1H-tetrazole-5-ylthio)butane-1-sulfonic acid.

Another aspect of the present invention provides a method of preparing the alkyl sulfonated tetrazole compound represented by Formula 1 above, comprising:

reacting tetrazole represented by Formula 2 below with alkyl sultone represented by Formula 3 below:

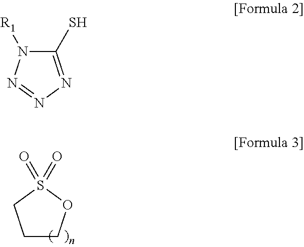

wherein R₁ and n were mentioned above.

Here, a molar ratio of the tetrazole represented by Formula 2 above to the alkyl sultone represented by Formula 3 above may be 1:0.5˜1.5.

Still another aspect of the present invention provides an epoxy resin comprising an alkyl sulfonated tetrazole compound, the epoxy resin being represented by Formula 4 below:

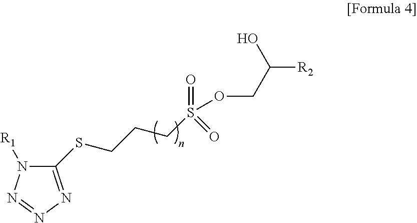

wherein R₁ is selected from an aliphatic or alicyclic alkyl group of 1 to 20 carbon atoms, an aryl or aralkyl group of 1 to 20 carbon atoms, an alkyl or aryl group substituted with a functional group of 1 to 20 carbon atoms, a ring linked with an alkylene group including or not including a heteroatom, a polymer compound group, and derivatives thereof; R₂ is a compound having one or more epoxy groups per molecule; and n is an integer of 1 to 6.

In the epoxy resin, R₂ may be at least one selected from the group consisting of bisphenol A, bisphenol F, bisphenol AD, catechol, polyphenol, polyglycidyl ether, glycidyl ether ester, polyglycidyl ester, epoxidized phenol novolac resin, epoxidized cresol novolac resin, epoxidized polyolefin, alicyclic epoxy resin, and urethane-modified epoxy resin.

Still another aspect of the present invention provides a method of preparing an epoxy resin containing the alkyl sulfonated tetrazole compound represented by Formula 4 above, comprising:

reacting the alkyl sulfonated tetrazole compound represented by Formula 1 above with an epoxy compound represented by Formula 5 below:

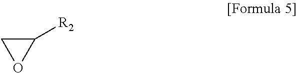

wherein R₁, R₂ and n were mentioned above.

Still another aspect of the present invention provides an epoxy resin composition including the epoxy resin containing the alkyl sulfonated tetrazole compound, the epoxy resin being represented by Formula 4 above.

The epoxy resin composition may further include at least one selected from the group consisting of bisphenol A, bisphenol F, bisphenol AD, catechol, polyphenol, polyglycidyl ether, glycidyl ether ester, polyglycidyl ester, epoxidized phenol novolac resin, epoxidized cresol novolac resin, epoxidized polyolefin, alicyclic epoxy resin, and urethane-modified epoxy resin.

Still another aspect provides a prepreg produced from the epoxy resin composition.

Still another aspect of the present invention provides a film produced from the epoxy resin composition.

Still another aspect of the present invention provides a substrate produced from the epoxy resin composition.

The substrate may include the prepreg produced from the epoxy resin composition.

The substrate may further include a metal layer located on one side or both sides of the prepreg.

The metal layer may have a pattern formed on one side or both sides thereof.

### DETAILED DESCRIPTION OF THE INVENTION

Before providing the detailed description of the present invention, the terminologies or words used in the description and the claims of the present invention should not be interpreted as being limited merely to their common and dictionary meanings. On the contrary, they should be interpreted based on the meanings and concepts of the invention in keeping with the scope of the invention based on the principle whereby the inventors can appropriately define the terms in order to describe the invention in the best way. Accordingly, it is understood that the form of my invention shown and described herein is to be taken as a preferred embodiment of the present invention and that various changes and modifications may be made in the invention without departing from the spirit and scope thereof.

Hereinafter, preferred embodiments of the present invention will be described in detail such that those skilled in the art can easily carry out the present invention.

As described above, the present invention provides an alkyl sulfonated tetrazole compound, a method of preparing the same, an epoxy resin containing the same and a substrate produced therefrom.

The alkyl sulfonated tetrazole compound according to the present invention is represented by Formula 1 below:

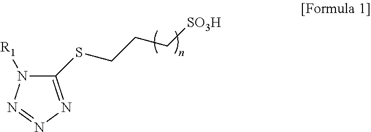

wherein R₁ is selected from an aliphatic or alicyclic alkyl group of 1 to 20 carbon atoms, an aryl or aralkyl group of 1 to 20 carbon atoms, an alkyl or aryl group substituted with a functional group of 1 to 20 carbon atoms, a ring linked with an alkylene group including or not including a heteroatom, a polymer compound group, and derivatives thereof, preferably, an aliphatic or alicyclic alkyl group of 1 to 10 carbon atoms, an aryl or aralkyl group of 1 to 10 carbon atoms, an alkyl or aryl group substituted with a functional group of 1 to 10 carbon atoms, a ring linked with an alkylene group including or not including a heteroatom, a polymer compound group, and derivatives thereof; and n is an integer of 1 to 6, preferably an integer of 3 to 4.

According to the present invention, as shown in Reaction Formula 1, in the case where tetrazole is reacted with an epoxy compound, when a thiol group (—SH) is directly bonded with the epoxy compound by ring-opening an epoxy group of the epoxy compound, the —S— group cannot donate electrons necessary to provide adhesion because of an hydroxy (OH) group adjacent thereto, so that the adhesion is unsatisfactory.

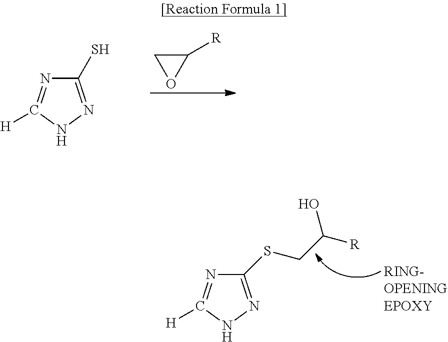

Here, R is an epoxy compound.

Therefore, the present invention is configured such that tetrazole represented by Formula 2 below is alkyl-sulfonated with an alkyl sultone represented by Formula 3 below to introduce a suitable sized alkyl group, thus allowing the —S— group to donate electrons necessary to provide adhesion.

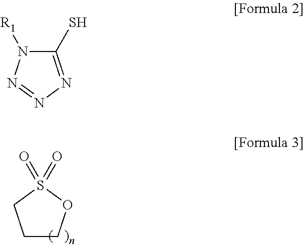

Here, R₁ and n were mentioned above.

That is, the method of preparing the alkyl sulfonated tetrazole compound represented by Formula 1 above is performed by Reaction Formula 2 in the present of a solvent. It is preferred that the molar ratio of the tetrazole represented by Formula 2 above to the alkyl sultone represented by Formula 3 above be 1:0.5˜1.5 stoichiometrically.

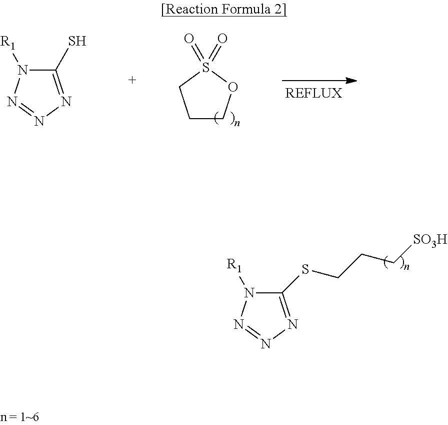

Here, R₁ and n were mentioned above.

It is preferred in terms of the adhesion of a final epoxy resin that the alkyl sulfonated tetrazole compound be 3-(1-methyl-1H-tetrazole-5-ylthio)propane-1-sulfonic acid or 3-(1-methyl-1H-tetrazole-5-ylthio)butane-1-sulfonic acid.

According to the present invention, the epoxy resin including the alkyl sulfonated tetrazole compound represented by Formula 1 above is represented by Formula 4 below.

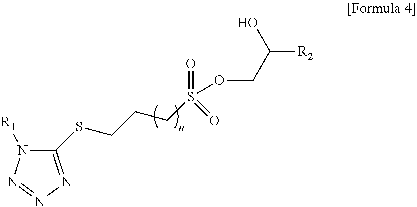

Here, R₁ is selected from an aliphatic or alicyclic alkyl group of 1 to 20 carbon atoms, an aryl or aralkyl group of 1 to 20 carbon atoms, an alkyl or aryl group substituted with a functional group of 1 to 20 carbon atoms, a ring linked with an alkylene group including or not including a heteroatom, a polymer compound group, and derivatives thereof; R₂ is a compound having one or more epoxy groups per molecule; and n is an integer of 1 to 6.

The epoxy resin, represented by Formula 4 above, including the alkyl sulfonated tetrazole compound is prepared by reacting the alkyl sulfonated tetrazole compound represented by Formula 1 above with an epoxy compound represented by Formula 5 below.

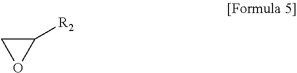

Here, R₂ was mentioned above.

This reaction is performed as shown in Reaction Formula 3 below in the presence of a solvent. It is preferred that the molar ratio of the alkyl sulfonated tetrazole compound to the epoxy compound be 1:0.5˜1.5.

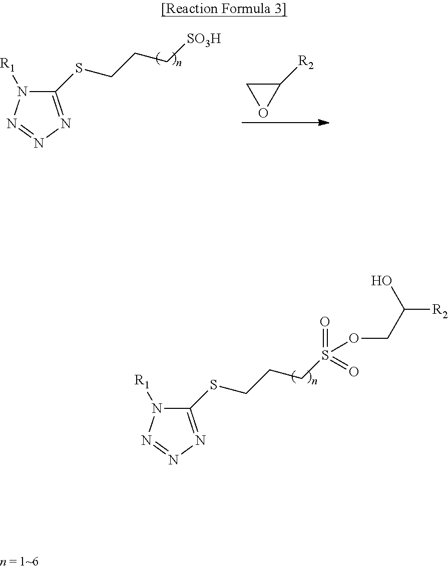

As shown in Reaction Formula 3 above, all or part of a functional group of the epoxy compound reacts with the alkyl sulfonated tetrazole compound represented by Formula 1 above to introduce tetrazole, which is a functional group for enhancing adhesion, to a resin, thus preparing a resin having high adhesion to a metal.

In the present invention, R₂ may be a compound having one or more epoxy groups per molecule. Examples of the compound may include: bisphenol A; bisphenol F; bisphenol AD; catechol, polyphenol such as resorcinol or the like; polyglycidyl ether obtained by reacting polyalcohol such as glycerin or polyethyleneglycol with epichlorohydrin; glycidyl ether ester obtained by reacting hydroxycarbonic acid such as p-hydroxybenzoic acid or β-hydroxynaphthoic acid with epichlrorohydrin; polyglycidyl ester obtained by reacting polycarbonic acid such as phthalic acid or terephthalic acid with epichlorohydrin; epoxidized phenol novolac resin; epoxidized cresol novolac resin; epoxidized polyolefin; alicyclic epoxy resin; and urethane-modified epoxy resin; and mixtures thereof. Preferably, example of the compound may include bisphenol A, bisphenol F, epoxidized phenol novolac resin, epoxidized cresol novolac resin, and the like.

Examples of the epoxy resin including the alkyl sulfonated tetrazole compound may include 3-(1-methyl-1H-tetrazole-5-ylthio)propane-1-sulfonyl cresol novolac resin, 3-(1-methyl-1H-tetrazole-5-ylthio)butane-1-sulfonyl cresol novolac resin, 3-(1-ethyl-1H-tetrazole-5-ylthio)propane-1-sulfonyl cresol novolac resin, 3-(1-ethyl-1H-tetrazole-5-ylthio)butane-1-sulfonyl cresol novolac resin, and the like. Preferably, the epoxy resin may be 3-(1-methyl-1H-tetrazole-5-ylthio)propane-1-sulfonyl cresol novolac resin represented by Formula 6 below.

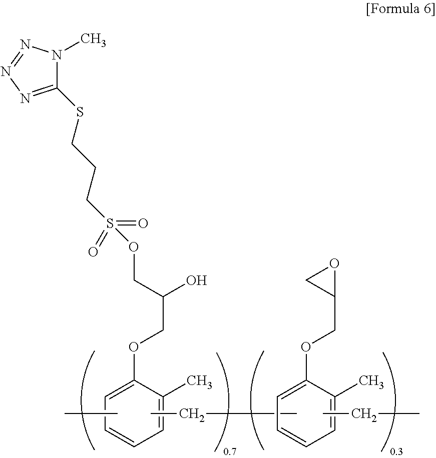

In the compound represented by Formula 6 above, the residual unreacted epoxy group can be completely substituted by adjusting an equivalent. However, in order to accelerate the curing reaction with other epoxy compounds and to maintain the adhesion attributable to tetrazole, it is effective that 1˜50% of the unreacted epoxy group exist.

Further, the present invention provides an epoxy resin composition including the epoxy resin, represented by Formula 4 above, containing the alkyl sulfonated tetrazole compound. The epoxy resin composition may further include a general epoxy compound. If necessary, the epoxy resin composition may further include a solvent, a curing agent and/or various kinds of other additives known to those skilled in the art. The other additives may include a filler, a softening agent, a plasticizer, an antioxidant, a flame retardant, an antistatic agent, a colorant, a thermal stabilizer, a light stabilizer, a UV absorber, a coupling agent, and an antiprecipitant.

The epoxy compound used in the epoxy resin composition may be at least one selected from the group consisting of bisphenol A, bisphenol F, bisphenol AD, catechol, polyphenol, polyglycidyl ether, glycidyl ether ester, polyglycidyl ester, epoxidized phenol novolac resin, epoxidized cresol novolac resin, epoxidized polyolefin, alicyclic epoxy resin, and urethane-modified epoxy resin.

The epoxy compound may be included in an amount of 0.1˜20 parts by weight based on 100 parts by weight of the epoxy resin compound. When the amount of the epoxy compound in the epoxy resin composition is 0.1˜20 parts by weight, the epoxy compound does not greatly influence the adhesion of the epoxy resin composition, and makes up for the mechanical and chemical properties of the epoxy resin composition depending on the characteristics of a resin.

The epoxy resin composition may be prepared by blending various components using various methods such as normal-temperature mixing, fusion mixing and the like.

The epoxy resin composition is cast on a substrate to form a thin film, and then the thin film is cured. The epoxy resin composition may be filtered using a filter or the like, and impurities may be removed from the epoxy resin composition before the epoxy resin composition is applied onto the substrate or impregnated into the substrate.

When a substrate is produced using the epoxy resin composition, the adhesion of the substrate to a metal can be improved.

Further, the present invention provides a prepreg produced from the above epoxy resin composition.

The prepreg can be produced by applying the epoxy resin composition onto a reinforcing material or impregnating the epoxy resin composition into the reinforcing material, curing the epoxy resin composition and then drying the epoxy resin composition to remove the solvent therefrom. Examples of the impregnation may include dip coating, roll coating and the like. Examples of the reinforcing material may include woven glass fiber cloth, woven alumina glass fiber cloth, nonwoven glass fiber cloth, nonwoven cellulose fabric, woven carbon fiber cloth, woven polymer cloth, and the like. Further, examples of the reinforcing material may include glass fiber, silica glass fiber, carbon fiber, alumina fiber, silicon carbide fiber, asbestos, rock wool, mineral wool, gypsum whister, and woven or nonwoven fabric thereof, aromatic polyamide fiber, polyimide fiber, liquid crystalline polyester, polyester fiber, fluorine fiber, polybenzoxazole fiber, glass fiber containing polyamide fiber, glass fiber containing carbon fiber, glass fiber containing polyimide fiber, glass fiber containing aromatic polyester, glass paper, mica paper, alumina paper, craft paper, cotton paper, glass-combined paper, and the like. They may be used as a mixture thereof. In this case, the thickness of the glass fiber cloth may be 5˜200 μm. The epoxy resin composition may be impregnated into the reinforcing material in an amount of 0.4˜3 parts by weight based on 1 part by weight of the reinforcing material. When the epoxy resin composition is impregnated within the above range, the adhesion between two or more prepregs is excellent, and the mechanical strength and dimensional stability of the prepreg are improved. The epoxy resin composition can be cured at a temperature of 150˜350° C., and the heat treatment thereof can be conducted at low temperature, thus manufacturing a printed circuit board.

The prepreg may be bonded with copper. That is, the prepreg may be bonded with copper by placing the prepreg produced in this way on copper foil and then heat-treating the prepreg. When the solvent is removed from the prepreg bonded with copper and heat treatment is carried out, a copper-bonded prepreg member is fabricated. In order to volatilize a solvent, heating may be conducted under reduced pressure or ventilation may be conducted. Examples of the coating method of the epoxy resin composition may include roller coating, dip coating, spray coating, spin coating, curtain coating, slit coating, screen coating, and the like.

Further, a solution of the epoxy resin composition can be formed into a film. Concretely, a solution layer of the epoxy resin composition is formed on a substrate by solvent casting, and then a solvent is removed from the solution layer, thus forming a film on the substrate. The substrate may be metal foil, such as copper foil, aluminum foil, gold foil, silver foil or the like, a glass substrate, a polyethylene terephthalate (PET) film or the like.

Another embodiment of the present invention provides a printed circuit board produced from the epoxy resin composition. The printed circuit board may be composed of a film, a copper clad laminate, a prepreg or a combination thereof. Further, the printed circuit board may be a copper clad laminate (CCL) or a flexible copper clad laminate (FCCL).

Further, the printed circuit board may include the above-mentioned prepreg. In this case, the printed circuit board may be produced by forming a metal layer on the prepreg, putting the prepreg into a press and then pressing and heating the prepreg to melt and cure the prepreg. The metal layer may be formed of copper, aluminum, iron, stainless, nickel or an alloy thereof. Further, the printed circuit board may be a printed circuit board in which metal layers are formed on both sides of prepreg, or may be a printed circuit board in which several prepreg layers have been pressed together. In addition, the printed circuit board including prepreg may be modified and used in various manners. Moreover, the printed circuit board may include conductive patterns formed on one side or both sides thereof or may include a multi-layer (4 layers, 8 layers or the like) conductive pattern.

Hereinafter, the present invention will be described in more detail with reference to the following Examples. However, the scope of the present invention is not limited to these Examples.

### Example 1

### Synthesis of 3-(1-methyl-1H-tetrazole-5-ylthio)propane-1-sulfonic acid

90 g of acetonitrile as a reaction solvent, 20 g (0.172 mol) of 5-mercapto-1-methyltetrazole and 21 g (0.172 mol) of 1,3-propane sultone were put into a 250 mL one-neck flask provided with a reflux condenser, and were then reacted at about 80° C. under a nitrogen atmosphere. The reaction ending point was determined by TLC analysis (developing solvent, chloroform:methanol=10:1), and thus the reaction was finished. Thereafter, the reaction product was filtered using Celite 545, and then the remainder was decompressed and concentrated to obtain 3-(1-methyl-1H-tetrazole-5-ylthio)propane-1-sulfonic acid by silica column chromatography. The yield thereof was 68%, and the melting point thereof was 98° C.

¹H-NMR (CDCl3, δ) 3.63 (s, 3H, tetrazole-CH₃), 3.40 (t, 2H, —S—CH₂CH₂CH₂—SO₃—) 2.95 (t, 2H, —S—CH₂CH₂CH₃—SO₃—), 2.20 (m, 2H, —S—CH₂CH₂CH₃—SO₃—).

### Example 2

### Synthesis of 3-(1-methyl-1H-tetrazole-5-ylthio)butane-1-sulfonic acid

3-(1-methyl-1H-tetrazole-5-ylthio)butane-1-sulfonic acid was obtained in the same manner as in Example 1, except that 23.4 g (0.172 mol) of 1,4-butane sultone was used instead of 21 g (0.172 mol) of 1,3-propane sultone. The yield thereof was 72%, and the melting point thereof was 105° C.

¹H-NMR (CDCl3, δ) 3.60 (s, 3H, tetrazole-CH₃), 3.41 (t, 2H, —S—CH₂CH₂CH₂CH₂—SO₃—) 2.92 (t, 2H, —S—CH₂CH₂CH₂CH₃—SO₃—), 1.72 (m, 2H, —S—CH₂CH₂CH₂CH₃—SO₃—), 1.83 (m, 2H, —S—CH₂CH₂CH₂CH₃—SO₃—)

### Example 3

100 g of methylethylketone (MEK) as a reaction solvent, 23.8 g (0.1 equivalents) of the 3-(1-methyl-1H-tetrazole-5-ylthio)propane-1-sulfonic acid obtained in Example 1 and 20.8 g (0.1 equivalents) of cresol novolac epoxy (YDCN500-80P) were put into a 250 mL one-neck flask provided with a reflux condenser, and were then stirred and reacted at about 80° C. under a nitrogen atmosphere. The reaction ending point was determined by analyzing acid value measurements, and thus the reaction was finished. Thereafter, the reaction product was precipitated in ethyl ether to obtain a tetrazole-substituted epoxy derivative. The obtained derivative is represented by Formula 6 above. The yield thereof was 85% (the rate of the substituted tetrazole is 70%), and the softening point thereof was 73° C.

¹H-NMR (CDCl3, δ) 3.66 (s, 3H, tetrazole-CH₃), 3.41 (t, 2H, —S—CH₂CH₂CH₂—SO₃—) 2.93 (t, 2H, —S—CH₂CH₂CH₃—SO₃—), 2.25 (m, 2H, —S—CH₂CH₂CH₃—SO₃—), 3.58 (m, 2H, —SO₃—CH₂CHCH₂—) 3.86 (m, 1H, —SO₃—CH₂CHCH₂—) 4.12 (m, 2H, —SO₃—CH₂CHCH₂—).

### Example 4

### Formation of an Insulating Film Containing Tetrazole-Substituted Epoxy

70 g of a bisphenol A type epoxy resin (YD-011, manufactured by KukDO Chemical Co., Ltd., epoxy equivalent: 469), 30 g of the tetrazole-substituted cresol novolac epoxy (the rate of substituted tetrazole: 70%) prepared in Example 3 and 250 g of spherical silica slurry (average particle size: 0.3 μm, dissolved in MEK at 65%) were mixed, and were then dispersed using a bead mill. Subsequently, 2 g of 2-ethyl-4-methylimidazole as a curing agent was added to the composition to prepare a resin varnish, and then the resin varnish was applied onto a polyethylene terephthalate (PET) film having a thickness of about 38 μm using a barcoater, and was then dried for about 10 minutes so that the thickness of the resin varnish becomes about 40 μm after it was dried.

### Comparative Example 1

100 g of a bisphenol A type epoxy resin (YD-011, manufactured by KukDO Chemical Co., Ltd., epoxy equivalent: 469) and 250 g of spherical silica slurry (average particle size: 0.3 μm, dissolved in MEK at 65%) were mixed, and were then dispersed using a bead mill. Subsequently, 2 g of 2-ethyl-4-methylimidazole as a curing agent was added to the composition to prepare a resin varnish, and then the resin varnish was applied onto a polyethylene terephthalate (PET) film having a thickness of about 38 μm using a barcoater, and was then dried for about 10 minutes so that the thickness of the resin varnish was about 40 μm after it was dried.

### Experimental Example

<Lamination of an Insulating Resin Sheet>

Each of the insulation films obtained in Example 4 and Comparative Example 1 was laminated by drawing the insulating film in a surface-roughened inner circuit board (conductor thickness: 18 μm, thickness thereof: 0.8 mm) by vacuum suction at about 80° C. for 20 seconds using a vacuum laminator and then pressing the insulation film for about 20 seconds under the conditions of a temperature of about 80° C. and a pressure of about 7.5 kg/cm².

<Curing of the Resin Composition>

A PET protective film was from the laminated insulation film, and then the insulation was cured at about 160° C. for about 30 minutes using a hot air furnace to obtain a laminate including an insulating layer formed on the section of the inner circuit board.

<Roughening>

The obtained laminate was surface-roughened using a permanganic acid solution. The process of surface-roughening the laminate is as follows. The laminate was immersed into a swelling treatment solution (swelling dip securiganth P, manufactured by Atotech Japan Corp.) at about 60° C. for about 10 minutes, and was then further immersed into an oxidation treatment solution (a mixed solution of a concentrate compact CP and dosing solution securiganth P) at about 80° C. for about 20 minutes. Thereafter, the laminate was immersed into a reduction treatment solution (reduction solution securiganth P500, manufactured by Atotech Japan Corp.) at about 40° C. for about 5 minutes.

<Formation of a Conductor Layer by Plating>

The roughened laminate was provided with a palladium catalyst on the surface thereof, and was then electroless-plated using a tartarate-containing printganth MSK-DK manufactured by Atotech Japan Corp. Thereafter, the laminate was electrolytically plated using copper sulfate such that the thickness of copper becomes about 20 μm. Finally, the electrolytically-plated laminate was cured at about 170° C. for about 50 minutes.

<Evaluation of Adhesion Strength>

The conductor layer formed by electrolytic plating was cut to a size of a width of 10 mm and a length of 100 mm, and then the adhesive strength thereof was evaluated at a speed of 50.8 mm/min and a length of 30 mm using a Z050 UTM (universal testing machine) manufactured by Zwick Corp. The results thereof are given in Table 1 below.

TABLE 1

| Class. | Adhesion strength (kg_{f}/cm) |
|---|---|
| Comparative Example 1 | 0.3 |
| Example 4 | 0.6 |

As given in Table 1 above, it can be seen that the epoxy resin including the alkyl sulfonated tetrazole compound exhibits the very excellent adhesive strength of about two times that of a general epoxy resin.

As described above, the alkyl sulfonated tetrazole compound and the epoxy resin including the same is effective to improve the adhesion to a metal when it is used in an interlayer insulation prepreg, a buildup film or the like.

Although a preferred embodiment of the present invention has been described for illustrative purposes, those skilled in the art will appreciate that various modifications, additions and substitutions are possible, without departing from the scope and spirit of the invention.

Accordingly, any and all modifications, variations or equivalent arrangements should be considered to be within the scope of the invention, and the detailed scope of the invention will be disclosed by the accompanying claims.

What is claimed is:

1. An alkyl sulfonated tetrazole compound represented by Formula 1 below: 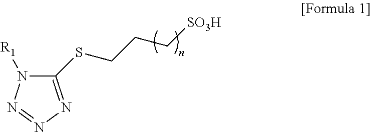
wherein R₁ is selected from an aliphatic or alicyclic alkyl group of 1 to 20 carbon atoms, an aryl or aralkyl group of 1 to 20 carbon atoms, an alkyl or aryl group substituted with a functional group of 1 to 20 carbon atoms, a ring linked with an alkylene group including or not including a heteroatom, a polymer compound group, and derivatives thereof; and n is an integer of 1 to 6.

2. The alkyl sulfonated tetrazole compound according to claim 1, wherein the alkyl sulfonated tetrazole compound is 3-(1-methyl-1H-tetrazole-5-ylthio)propane-1-sulfonic acid or 3-(1-methyl-1H-tetrazole-5-ylthio)butane-1-sulfonic acid.

3. A method of preparing an alkyl sulfonated tetrazole compound represented by Formula 1 below, comprising:
reacting tetrazole represented by Formula 2 below with alkyl sultone represented by Formula 3 below: 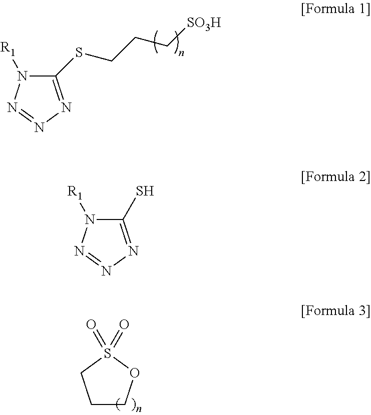
wherein R₁ is selected from an aliphatic or alicyclic alkyl group of 1 to 20 carbon atoms, an aryl or aralkyl group of 1 to 20 carbon atoms, an alkyl or aryl group substituted with a functional group of 1 to 20 carbon atoms, a ring linked with an alkylene group including or not including a heteroatom, a polymer compound group, and derivatives thereof; and n is an integer of 1 to 6.

4. The method according to claim 3, wherein a molar ratio of the tetrazole represented by Formula 2 above to the alkyl sultone represented by Formula 3 above is 1:0.5˜1.5.

5. An epoxy resin comprising an alkyl sulfonated tetrazole compound, the epoxy resin being represented by Formula 4 below: 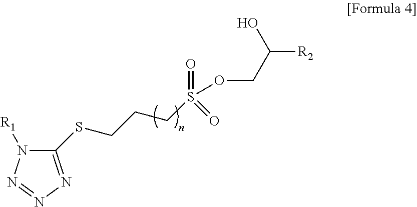
wherein R₁ is selected from an aliphatic or alicyclic alkyl group of 1 to 20 carbon atoms, an aryl or aralkyl group of 1 to 20 carbon atoms, an alkyl or aryl group substituted with a functional group of 1 to 20 carbon atoms, a ring linked with an alkylene group including or not including a heteroatom, a polymer compound group, and derivatives thereof; R₂ is a compound having one or more epoxy groups per molecule; and n is an integer of 1 to 6.

6. The epoxy resin according to claim 5, wherein R₂ is at least one selected from the group consisting of bisphenol A, bisphenol F, bisphenol AD, catechol, polyphenol, polyglycidyl ether, glycidyl ether ester, polyglycidyl ester, epoxidized phenol novolac resin, epoxidized cresol novolac resin, epoxidized polyolefin, alicyclic epoxy resin, and urethane-modified epoxy resin.

7. The epoxy resin according to claim 5, wherein the epoxy resin includes 1˜50% of an unreacted epoxy group.

8. A method of preparing an epoxy resin containing an alkyl sulfonated tetrazole compound represented by Formula 4 below, comprising:
reacting an alkyl sulfonated tetrazole compound represented by Formula 1 below with an epoxy compound represented by Formula 5 below: 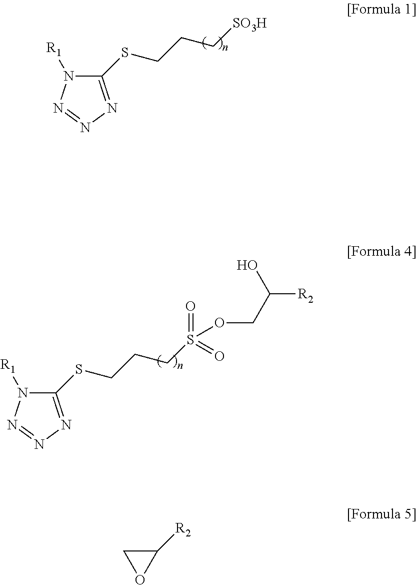
wherein R₁ is selected from an aliphatic or alicyclic alkyl group of 1 to 20 carbon atoms, an aryl or aralkyl group of 1 to 20 carbon atoms, an alkyl or aryl group substituted with a functional group of 1 to 20 carbon atoms, a ring linked with an alkylene group including or not including a heteroatom, a polymer compound group, and derivatives thereof; R₂ is a compound having one or more epoxy groups per molecule; and n is an integer of 1 to 6.

9. The method according to claim 8, wherein a molar ratio of the alkyl sulfonated tetrazole compound to the epoxy compound is 1:0.5˜1.5.

10. The method according to claim 8, wherein the epoxy resin includes 1˜50% of an unreacted epoxy group.

11. An epoxy resin composition comprising the epoxy resin containing an alkyl sulfonated tetrazole compound according to claim 5.

12. The epoxy resin composition according to claim 11, further comprising at least one epoxy compound selected from the group consisting of bisphenol A, bisphenol F, bisphenol AD, catechol, polyphenol, polyglycidyl ether, glycidyl ether ester, polyglycidyl ester, epoxidized phenol novolac resin, epoxidized cresol novolac resin, epoxidized polyolefin, alicyclic epoxy resin, and urethane-modified epoxy resin.

13. A prepreg produced from the epoxy resin composition of claim 11.

14. A film produced from the epoxy resin composition of claim 11.

15. A substrate produced from the epoxy resin composition of claim 11.

16. The substrate according to claim 15, comprising the substrate comprises the prepreg produced from the epoxy resin composition.

17. The substrate according to claim 16, further comprising a metal layer located on one side or both sides of the prepreg.

18. The substrate according to claim 17, wherein the metal layer has a pattern formed on one side or both sides thereof.
